(19) United States
(12) Patent Application Publication
(10) Pub. No.: US 2003/0073071 A1
(43) Pub. Date: Apr. 17, 2003

(54) Solid state sensing system and method for measuring the binding or hybridization of biomolecules

(76) Inventors: Jurgen Fritz, Cambridge, MA (US); Emily Cooper, Cambridge, MA (US); Scott Manalis, Santa Barbara, CA (US)

Correspondence Address: TESTA, HURWITZ & THIBEAULT, LLP, HIGH STREET TOWER, 125 HIGH STREET, BOSTON, MA, 02110, US

(21) Appl. No.: 10/201,333

(22) Filed: Jul. 23, 2002

Related U.S. Application Data
(60) Provisional application No. 60/329,204, filed on Oct. 12, 2001.

(51) Int. Cl. C12Q 1/00; C12M 1/34

(52) U.S. Cl. 435/4; 435/287.2; 510/179

(57) ABSTRACT

Measurements relating to the activity of various molecules of interest are obtained using a sensing surface with an associated first charge, a charge-regulating layer bound to the sensing surface, various possible probe molecules and a measurement circuit. The sensing surface has a layer bound to it that confers a neutral charge or a second charge on a net basis. In addition, a probe is bound to at least one of the surface and the layer, the probe being complementary to and interacting with a molecule of interest. The interaction between the probe and the molecule of interest is detected electrically.

### CROSS-REFERENCE TO RELATED APPLICATIONS

This application claims the benefits of provisional U.S. Patent Application Serial No. 60/329,204 filed on Oct. 12, 2001, the disclosure of which is hereby incorporated herein by reference in its entirety.

### FIELD OF THE INVENTION

The present invention relates generally to the field of solid state sensing systems and, more specifically, to sensing systems and methods for measuring the properties of biomolecules.

### BACKGROUND OF THE INVENTION

The desire to detect and compare the binding and hybridization properties of various biomolecules is a longstanding goal of scientific researchers. A variety of optical, chemical, fluorescent, pH-based, and electrical techniques have been developed in an attempt to achieve these goals. Given the critical importance of research in this area for human health and welfare, innovation in this field is of great value.

An important criterion for such techniques is the speed at which experimental data can be acquired and processed. Therefore experiments, which may be performed in a fraction of the time as was previously considered the standard in that field, are of great scientific and financial value. In addition, given that the high costs of many experimental reagents constrain the frequency or perhaps even the possibility of performing certain experiments, a reduction in experimental size which reduces reagent volume can bring down costs.

At the same time, the complexities of surface interaction chemistry pose a critical challenge in designing experimental and measurement equipment. Surface features and reactivity impact, for example, the kinetic interaction of molecules in solution, the covalent interaction of biomolecules at an adsorption layer, and the shielding effects of various compounds in solution. These, in turn, can all affect the instrumentation design process.

In particular, the ionic strength of a sample solution directly affects detector sensitivity due to counter-ion screening. At high ionic strengths, counter-ions shield the charge characteristics of a molecule of interest, interfering with their detection. As a result, detector systems are generally operated using low-ionic-strength environments. The price to be paid, however, is reduced reaction kinetics, i.e., lengthening of reaction times. This tradeoff between sensitivity and time-scale is a persistent difficulty in the design of chemical sensing systems.

### SUMMARY OF THE INVENTION

The present invention operates to reduce surface interactions that interfere with biomolecular sensing, thereby increasing the sensitivity with which sensing can be carried out. Moreover, by reducing the effects of screening and, in effect, creating a localized environment facilitating fast reaction kinetics, the invention achieves this sensitivity improvement without concomitant sacrifice of reaction speed. Accordingly, within limits, the invention addresses the fundamental tradeoff between sensitivity and speed.

The measuring apparatus of the present invention measures and detects electrical changes such as voltage, current, and/or capacitance that result from biomolecular interactions with the sensing surface. Capacitive sensing represents a preferred embodiment. The invention may be fabricated to withstand the application of strong cleaning agents in order to facilitate repeated use of the apparatus with varying chemical samples. Moreover, the invention can be employed in microfluidic channels as a result of the size and geometry of various embodiments. The invention facilitates the rapid and selective detection of label-free biomolecules without necessitating the use of traditional chemical amplification techniques. In general, the methods of the invention for hybridizing biomolecules of interest on a charge-compensated surface at low ionic strength can also be extended to other field-effect sensing devices, such as silicon nanowires.

In one aspect, the invention comprises an apparatus for measuring biomolecular interaction. The apparatus includes a sensing surface with an associated first charge, and a charge-regulating layer bound to the sensing surface. The charge-regulating layer has an associated second charge opposite to the first charge. The charge-regulating layer, in conjunction with the sensing surface, presents the second charge or a neutral charge on a net basis. This aspect of the invention can further include a probe bound to at least one of the sensing surface or the charge-regulating layer. The probe is chosen so as to be complementary to and interacting with a molecule of interest. A measurement circuit may be operatively connected to the sensing surface, and measures interactions between the probe and the molecule of interest.

The sensing surface of the invention is silicon dioxide in various preferred embodiments. The probe can include, for example, a nucleic acid, a polypeptide, a protein nucleic acid, a substrate interactive with a polypeptide, an enzyme interactive with a substrate, an antibody interactive with an antigen, an antigen interactive with one or more antibodies, or other biomolecule. The apparatus can be passivated to retain operational functionality notwithstanding cleaning in various preferred embodiments.

In some embodiments, a native negative charge is neutralized or rendered positive by the charge-regulating layer, which may comprise, for example, polylysine. When polylysine is chosen for the charge-regulating layer it can become electrostatically bound to the sensing surface. In various embodiments, the invention is configured so the sensing surface is an input for the gate of a field-effect transistor.

The interaction of the probe and molecule of interest generates an associated electrical response in the measurement circuit in various embodiments. The magnitude of this electrical response is correlated with the degree of interaction between the probe and the molecule of interest. For example, the interaction between the probe and the molecule of interest may alter a capacitance within the measurement circuit, the alteration indicating the level of interaction.

The measurement circuit can include a charge-sensitive region underlying the sensing surface, and a semiconductor region at least partially surrounding the charge-sensitive region. In various preferred embodiments, the sensing surface, the charge-sensitive region and the semiconductor region form at least one capacitor.

In some embodiments, the charge-sensitive region and at least a portion of the semiconductor region are fabricated to form a cantilever. The cantilever can be configured for insertion into microfluidic channels. In other embodiments, the charge-sensitive region and at least a portion of the semiconductor region form a plurality of cantilevers electrically connected to facilitate differential measurements of the properties of molecules of interest.

In still another aspect the invention comprises a method of measuring biomolecular interaction. This method includes the steps of initially providing a sensing surface having a native charge. The sensing surface provided has a charge-regulating layer bound to it that confers to the sensing surface a neutral or opposite charge. In addition, a probe is bound to at least one of the surface and the charge-regulating layer, the probe being complementary to and interacting with a molecule of interest. The interaction between the probe and the molecule of interest is then measured.

In yet another aspect, a method in accordance with the invention includes providing a sensing surface comprising a probe complementary to and interacting with a molecule of interest; and capacitively measuring the interaction between the probe and the molecule of interest.

### BRIEF DESCRIPTION OF THE DRAWINGS

The invention is pointed out with particularity in the appended claims. The advantages of the invention described above, together with further advantages, may be better understood by referring to the following description taken in conjunction with the accompanying drawings. In the drawings, like reference characters generally refer to the same parts throughout the different views. The drawings are not necessarily to scale, emphasis instead generally being placed upon illustrating the principles of the invention.

FIG. 1 is a schematic cross-section of a measuring apparatus according to an illustrative embodiment of the invention;

FIG. 2 is a schematic diagram depicting chemical interactions occurring at a sensing surface according to an illustrative embodiment of the invention;

FIG. 3 is a graph depicting the effect on the surface potential of the sensing surface upon alternately adding charge-regulating layers and probe molecules according to an illustrative embodiment of the invention;

FIG. 4 is a diagram depicting a capacitively based measuring apparatus in a cantilever configuration according to an illustrative embodiment of the invention;

FIG. 5 depicts a transistor-based measuring apparatus according to an illustrative embodiment of the invention;

FIG. 6 is a graph depicting the effect of electolyte pH on the surface potential of the sensing surface; and

FIG. 7 is a plan view of a measuring apparatus in a dual cantilever configuration suitable for microfluidic channel insertion according to an illustrative embodiment of the invention.

### DESCRIPTION OF THE PREFERRED EMBODIMENTS

Embodiments of the present invention are described below. It is, however, expressly noted that the present invention is not limited to these embodiments, but rather the intention is that modifications that are apparent to the person skilled in the art and equivalents thereof are also included.

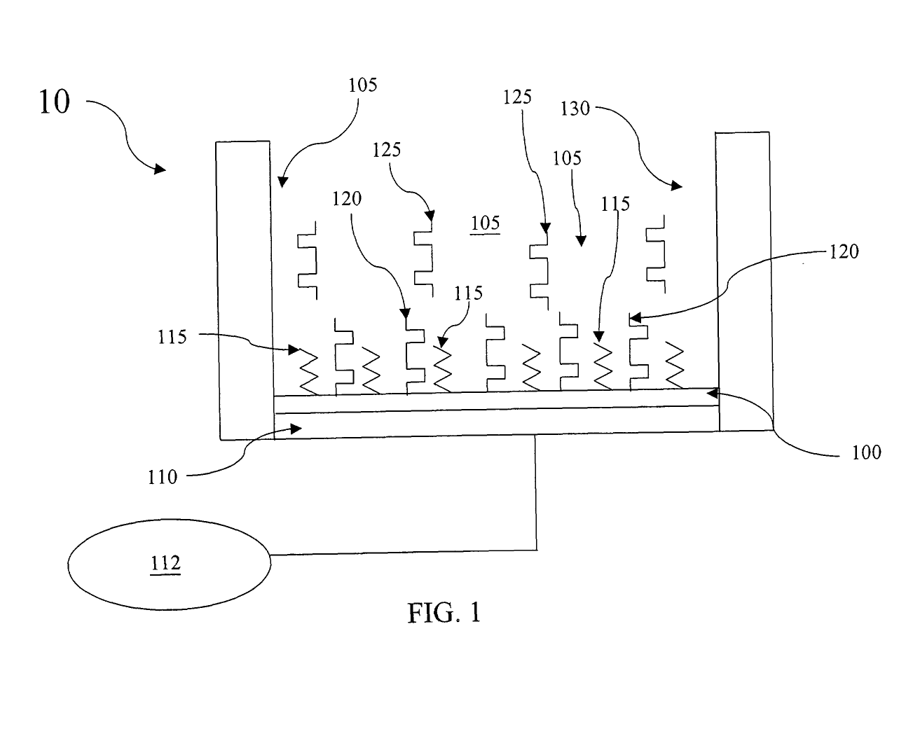

Referring to FIG. 1, various features which comprise a measuring apparatus embodiment 10 in accord with the invention are illustrated. A sensing surface 100 resides within a sample-containing region 105. The sample-containing region 105 can be fabricated to define any structure suitable for fluid containment.

The sensing surface 100 has an associated electrical charge. This charge typically arises from the intrinsic properties of the material(s) comprising the sensing surface 100. The sensing surface 100 may, for example, comprise an oxide such as silicon dioxide. In such embodiments, the negatively charged oxygen molecules impart a net negative charge to the sensing surface 100.

Underlying the sensing surface 100 are one or more electrically responsive layers, representatively indicated at 110 and described in greater detail below, which sense molecular interactions occurring on sensing surface 100. A measurement circuit 112 detects the electrical response and, hence, the degree and/or character of the interaction.

The sample-containing region 105 receives chemical solutions, which can include constituents of a charge-regulating layer 115, probe molecules 120, and various molecules of interest 125. In various embodiments, the sample containing region 105 is filled with a buffer solution. The buffer solution is preferably an electrolyte, such as 10 mM HEPES(2-[4-(2-hydroxyethyl)-1-piperazinyl]ethanesulfonic acid), and 5 mM NaCl. In other embodiments, the sensing surface 100 and the surrounding components of the measuring apparatus 10 can be immersed in a separate container or microfluidic channel comprising the buffer solution and various analytes of interest.

Constituents of the charge-regulating layer 115 may be introduced into the sample containing region 105 in combination with the buffer solution, and bind electrostatically or covalently to the sensing surface 100, thus forming a continuous or, more typically, discontinuous layer thereover. These constituents have an electrical charge associated with them. This charge is generally opposite to the native charge of the sensing surface 100. In various preferred embodiments, the charge-regulating layer comprises a positively charged species, generally a charged polymer such as poly-L-lysine (PLL), aminosilane, divalent ions or combinations thereof. Charge-regulating layers 115 have been deposited, for example, via buffer solutions comprising 0.1 mg/mL PLL (25 kDa poly-L-lysine•HCl) and buffer streams with 5 μg/mL.

Once the charge-regulating layer 115 is established, probe molecules 120 may be introduced into the sample containing region 105 of the measuring apparatus 10. The probe molecules 120 are chosen based on their affinity for a given molecule of interest 125, the probe molecule's charge characteristics, and the properties of the charge-regulating layer 115 being employed. Probe molecules 120 may be associated with the sensing surface 100 simply through addition to the solution within the sample-containing region 105. The probe molecules 120 bind electrostatically or covalently to the charge-regulating layer 115 and/or to the sensing surface 100. When the molecules of interest 125 are subsequently introduced into the sample-containing region 105, they bind to the previously introduced probe molecules, and the measurement apparatus 10 detects electrical changes as a result of the molecule of interest's attachment to a probe molecule 120. Compounds suitable for use as probes 120, or molecules of interest 125, include, for example, peptide nucleic acids, deoxyribonucleic acids, ribonucleic acids, proteins, polypeptides, amino acids and enzymes. In particular, if the molecule of interest is an enzyme present in the sample, the probe may be a substrate for the enzyme; conversely, the probe may be an enzyme for a substrate of interest in the sample. Similarly, the probe may be an antibody specific for an antigen in the sample, or conversely, may be an antigen complementary to one or more antibodies in the sample. Chemistries for binding such probe molecules to a sensing surface 100 or a charge-regulating layer 115 are conventional and well-characterized in the art.

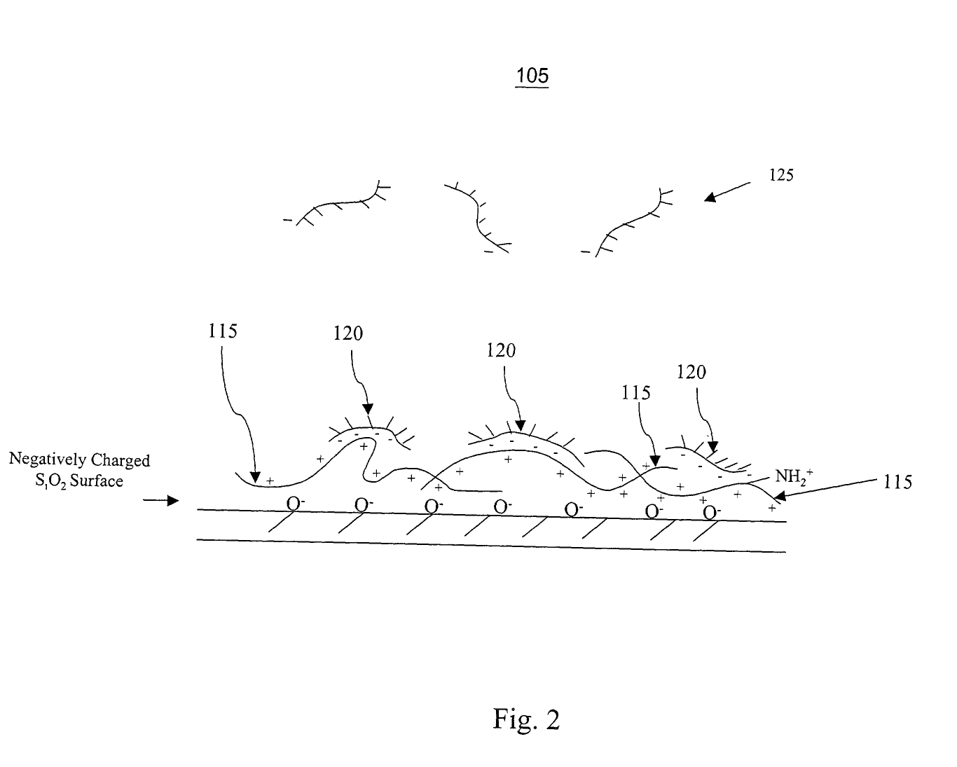

Referring to FIG. 2, a molecular level view of the interactions of various compounds at the sensing surface 100 of an illustrative embodiment of the invention are shown. The sensing surface in this embodiment comprises silicon dioxide. The negatively charged oxygen molecules give the sensing surface 100 an overall negative charge. Constituents of the charge-regulating layer 115 have been introduced into the sample-containing region 105 and allowed to intermittently bind to the sensing surface 100. The degree to which the charge-regulating layer 115 binds to the sensing surface 100 can vary based upon experimental conditions such as temperature and ionic strength of the buffer solution. In this embodiment the charge-regulating layer 115 is positively charged and comprises, for example, PLL. The probe molecules 120 and the molecules of interest 125 may be, for example, complementary nucleic acids. Both the probe molecules 120 and the molecules of interest 125 present in this illustrative embodiment are negatively charged. Consequently, the probe molecules 120 bind electrostatically to the charge-regulating layer 115. If the gross positive charge exhibited by the species 115 exceeds the net negative charge of the surface 100, then the surface 100 will present a net positive charge; in general, it is desirable to achieve at least a neutral state, and more typically, in the case of negatively charged probe molecules, a net positive charge. When the molecule of interest 125 for a given experiment is introduced into the sample-containing region 105 it can readily bind to or hybridize with the complementary probe molecule 120 because of the screening effect of the charge-regulating layer 115.

The initial binding of the charge-regulating layer 115 and the subsequent binding of the probe molecules 120 to the charge-regulating layer 115 causes electrical changes at the sensing surface 100 which can be measured and distinguished from one another. Binding of the molecules of interest 120 causes further electrical changes at the sensing surface 100 which can be measured through capacitive, current, or voltage-based means.

By ameliorating the interference with bonding caused by screening, the present invention improves reaction kinetics at a given concentration of probe molecules 120. As a result, high-ionic-strength conditions which can reduce detection sensitivity are not necessary to achieve desirably fast reaction rates.

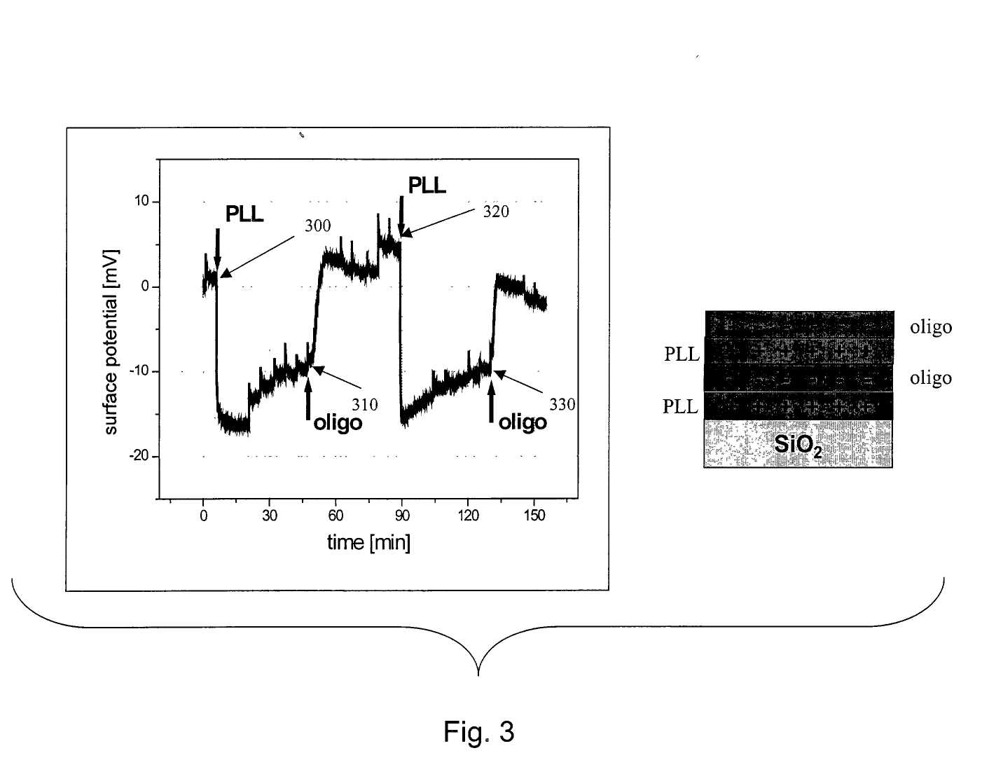

Referring to FIG. 3, the shortened experimental timescale required for experiments using a device as described above, as well as the electrical effects of binding PLL to the sensing surface 100 are illustrated. The shortened experimental timescale is achieved at low-ionic-strength concentrations where field-effect detection is most sensitive. As shown in the graph, the initial introduction 300 of positively charged PLL results in reduction of the surface potential as the PLL bonds to the silicon dioxide sensing surface. When a negatively charged oligonucleotide (specifically, a 12 mer) is introduced at 310, the surface potential rises as the oligonucleotide binds to the PLL and to the sensing surface 100. The second introduction 320 of PLL demonstrates the same surface-potential-reducing effect as the first PLL introduction 300. The second introduction 330 of the oligonucleotide once again increases the surface potential. Thus, this process of charge regulation through a charge-regulating layer 115 and a probe molecule 125 is repeatable on the same device.

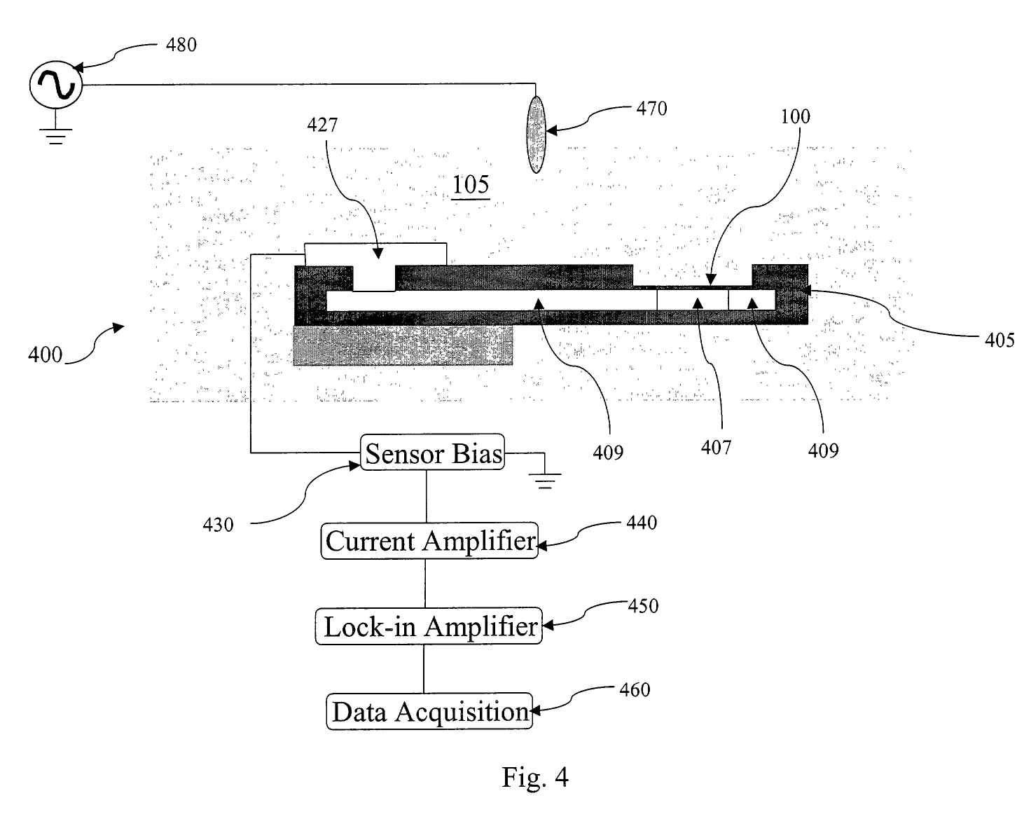

FIG. 4 illustrates a preferred device 400 in accordance with the invention, which uses capacitive measurements to detect molecular binding and hybridization events. The illustrated device comprises a sample-containing region 105 containing a buffer solution, the sensing surface 100, and various measurement circuit components. The positively charged charge-regulating layer operates to increase measurement sensitivity. The measurement device 400 is configured as a cantilever. The cantilever configuration is particularly suitable for insertion into the sample containing microfluidic channels.

For example, the device 400 may be utilized by insertion into a microfluidic channel containing a buffer solution. In one embodiment, the device 400 operates as a scanning probe potentiometer (SPP). The SPP is itself a field-effect device that includes an electrolyte-insulator-semiconductor (EIS) structure. This capacitive structure achieves a surface potential resolution similar to that of ion-sensitive field-effect transistors (ISFETs), but requires only one electrical connection, a contact electrode, to the silicon portion of the device. The illustrated SPP embodiment 400 includes an insulating shell 405, which may be silicon dioxide, and upon which the sensing surface 100 is defined. A charge-sensitive depletion region 407 (e.g., lightly doped silicon) underlies the sensing surface 100. Surrounding the charge-sensitive region 407 on all sides (but not on the top and bottom surfaces, which are surrounded by silicon dioxide as shown) is a semiconductor region 409 which can comprise highly doped silicon. A contact (e.g., aluminum) electrode 427 serves as the external connection point for the measurement circuit, contacting the insulator region 405 and the semiconductor region 409. It is through the contact with semiconductor region 409 that the electrode 427 receives the electrical changes at the sensing surface 100 that are imparted to the charge-sensitive region 407. The contact electrode 427 is connected to a sensor bias module 430, a current amplifier 440, a lock-in amplifier 450, and a data-acquisition module 460. A reference electrode 470, which is driven by an AC source 480, is immersed in the sample solution (and therefore accompanies a cantilever device 400 into the microfluidic channels into which it is inserted). Various capacitive elements exist in the device 400: between the reference electrode 470 and the electrolyte buffer (i.e., sample) solution; between the buffer solution and insulator 405; and between the charge-sensitive region 407 and the semiconductor region 409. Interaction between the probe and the molecule of interest alters the overall capacitance of the device 400, the degree of alteration being indicative of the interaction.

The extent of the depletion region in the silicon portion of the device 400, which provides a measurement of the potential at the electrolyte-insulator interface, is typically measured in two ways. The first, exemplified by the light-addressable potentiometric sensor (LAPS), measures the current of photogenerated carriers drifting in the high electric field of the depletion region. The second technique measures the depletion region capacitively, by applying a small alternating current to the bias voltage. The capacitive measurement is preferred. The SPP miniaturizes the EIS concept from the millimeter scale to the micrometer scale and facilitates it integration onto an atomic force microscope cantilever.

The device 400 can be fabricated at sizes sufficiently small to profile the pH of nanoliter volumes in a plurality of discrete solutions. We have, for example, submerged an entire cantilever in microfluidic channels with widths below 100 μm. The cantilever design allows the sensor to be rapidly (<1 s) scanned through many analytes which themselves remain in spatially distinct locations (i.e., different channels). This enables measurement of reaction dynamics without complications of volume exchange within a fixed chamber, including adsorptive losses to chamber walls and mechanical disturbances.

The application of field-effect sensors to bioanalytical tasks makes evident the need for an EIS sensor that is not only reliable during operation in electrolyte solutions, but also robust to a wide variety of stringent cleaning procedures necessary to run multiple experiments with a single device. Drawing from integrated-circuit design methodology, previous devices relied on metal electrical traces passivated by plasma enhanced chemical vapor deposition (PECVD) oxide and nitride films. Unfortunately, cleaning with acids can exacerbate defects in imperfect PECVD passivation films, making it difficult to repeat experiments reliably.

In one embodiment a device 400 is fabricated based on a heavily doped silicon electrical trace passivated with thermally grown silicon dioxide. This device can withstand aggressive cleaning procedures and can reliably monitor the adsorption of PLL and its operation as a charge-regulating layer. The robustness of such devices has been demonstrated by measuring similar current-voltage characteristics before and after the entire measurement device 400 is cleaned in 3:1 H₂SO₄:H₂O₂ (“piranha”). The performance of such devices has been reliable through multiple experiments and cleaning cycles for more than 90 days, providing robust and reproducible surface charge detection.

Measurement devices in accordance with the present invention can be fabricated using various techniques. To manufacture a series of the cantilever embodiment shown in FIG. 4, processing may begin with double-side-polished silicon-on-insulator (SOI) substrates. The device layer is initially 2.2 μm thick p-type (boron-doped) silicon with 4-6 Ω-cm resistivity, and the buried oxide layer is 1.1 μm thick. After masking a small area for the charge sensitive region 407, the rest of the wafer is implanted with boron to achieve a relatively uniform doping level of ˜10¹⁸ atoms/cm³ after the anneal. Electrical traces are defined by patterning the highly doped silicon region. The mask is removed, and a thick (1.1 μm) thermal oxide is grown to passivate the device. The thick oxide is then cleared from the charge sensitive region of the SPP through, for example, exposure to hydrofluoric acid (HF), and replaced with a 100 nm layer of thermally diffused oxide, which forms the final sensing surface 100 of the charge sensitive regions. Next, contact cuts are made in the die, and aluminum is deposited and patterned to make contacts 427. Finally, the finished devices are released with a deep reactive ion etch.

In other embodiments, the entire oxide coating is removed (e.g., HF is used to etch down to the silicon), following which the device is exposed to a medium that promotes growth of a thin insulating layer, e.g., a chemical oxide or other chemical passivation, on the silicon sensing surface. For example, cleaning in piranha solution is found to promote the growth of such a layer. If the probe and, if desired, the charge-regulating species are thereupon applied to the chemically passivated surface, a device of particularly high sensitivity is obtained.

The active sensing areas in thus-manufactured devices may range from 5 nm on a side to 100 nm on a side, and demonstrate similar surface potential resolution over the range of sensors. In some embodiments, the devices have a 50×50 gm² sensing region covered with a 2 nm layer of oxide. This size may itself contribute to enhanced sensitivity relative to prior-art devices, since a small sensing area will contain a relatively small population of sensitivity-affecting defects.

The thin layer of oxide facilitates detection of molecular charges by field-effect measurements because the electric field decays inversely with the thickness of the insulating oxide. In a closed fluidic chamber, 50 μV changes are observable in a 1 Hz bandwidth, commensurate with the demonstrated resolution of commercial LAPS devices. The thickness of the passivation oxide and the high doping level of the electrical trace guarantee that the response of the measuring device is dominated by the response of the lightly doped charge-sensitive region 407.

In some embodiments, a Ag/AgCl wire is used as the counter (reference) electrode 470 in solution. The measuring apparatus is typically biased via the sensor bias module 430 such that the lightly doped silicon of the charge-sensitive region 407 is depleted to about half of its maximum depletion depth, where the capacitive response to surface potential changes is linear and most sensitive. A 0.1 V AC signal at 1-10 kHz is applied to the bias voltage output by bias module 430 to generate a charging current through the AC source 480. The charging current is amplified by the current amplifier 440 and then its root-mean-square (RMS) amplitude is monitored by the lock-in amplifier 450. Gain and offset may be adjusted with a differential amplifier and captured by the data acquisition module 560 (which is typically a computer programmed for data capture).

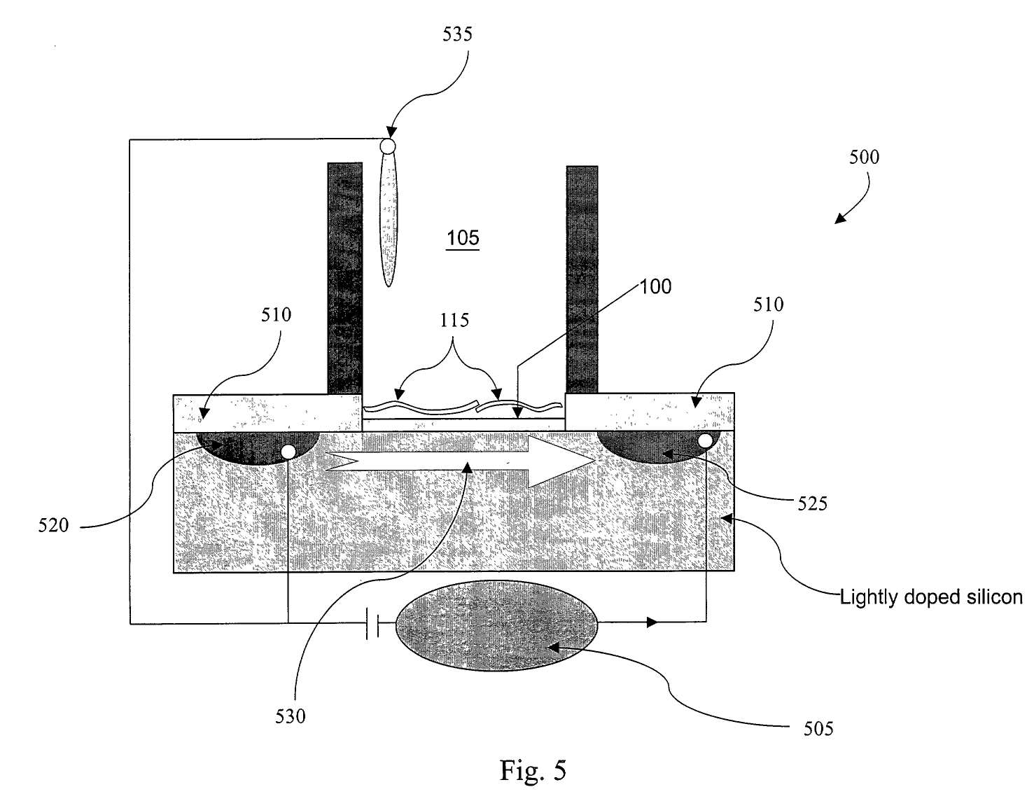

FIG. 5 illustrates another embodiment of the invention configured as a chemically responsive field-effect transistor (i.e., a chemFET). In particular, FIG. 5 illustrates the relationship between this device and a more conventional chemFET. The illustrated device 500 comprises a sample-containing region 105, a sensing surface 100, and a measurement circuit 505. The positively charged charge-regulating layer 115 operates to increase the measuring apparatus sensitivity. The sensing surface 100 is once again defined on an insulating layer 510. Underlying layer 510 and flanking sensing surface 100 are a source element 520 and a drain element 525. An electron channel 530 runs between the source 520 and the drain 525. Molecular interactions occurring on the sensing surface 100 affect the movement of charge in the election channel 530. A reference electrode 535 is immersed in the sample solution, and the source-to-drain current (which is indicative of the molecular interactions under study) is measured by the circuit 505.

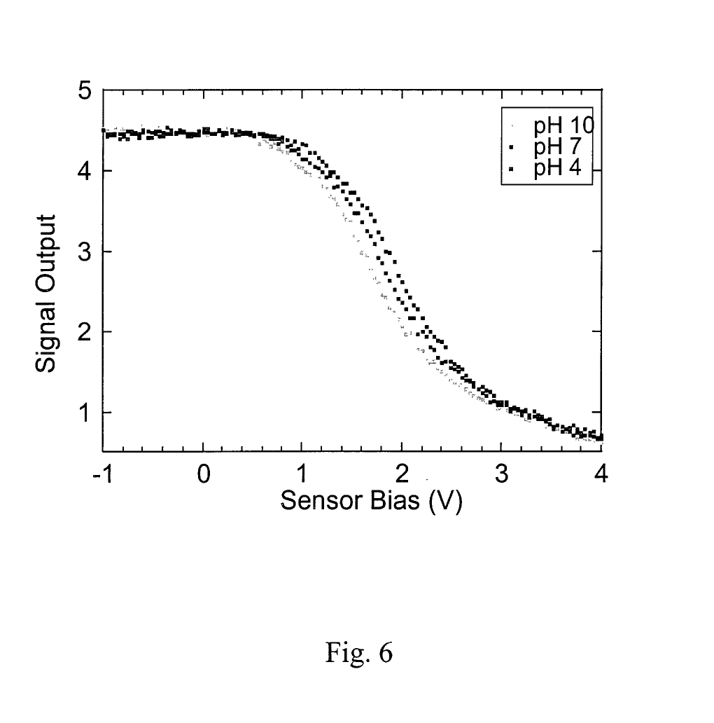

The relationship between ionic condition strength, surface potential bias and signal output for an SPP embodiment is further illustrated in the graph shown in FIG. 6. The pH of 4 corresponds to an increase of H⁺ in solution, and the pH of 10 corresponds to a decrease in H⁺ in solution. The graph in FIG. 6 shows that as more H⁺ is available for binding to the silicon dioxide surface, the oxide surface potential increases. The pH response of some embodiments is approximately 16 mV/pH unit.

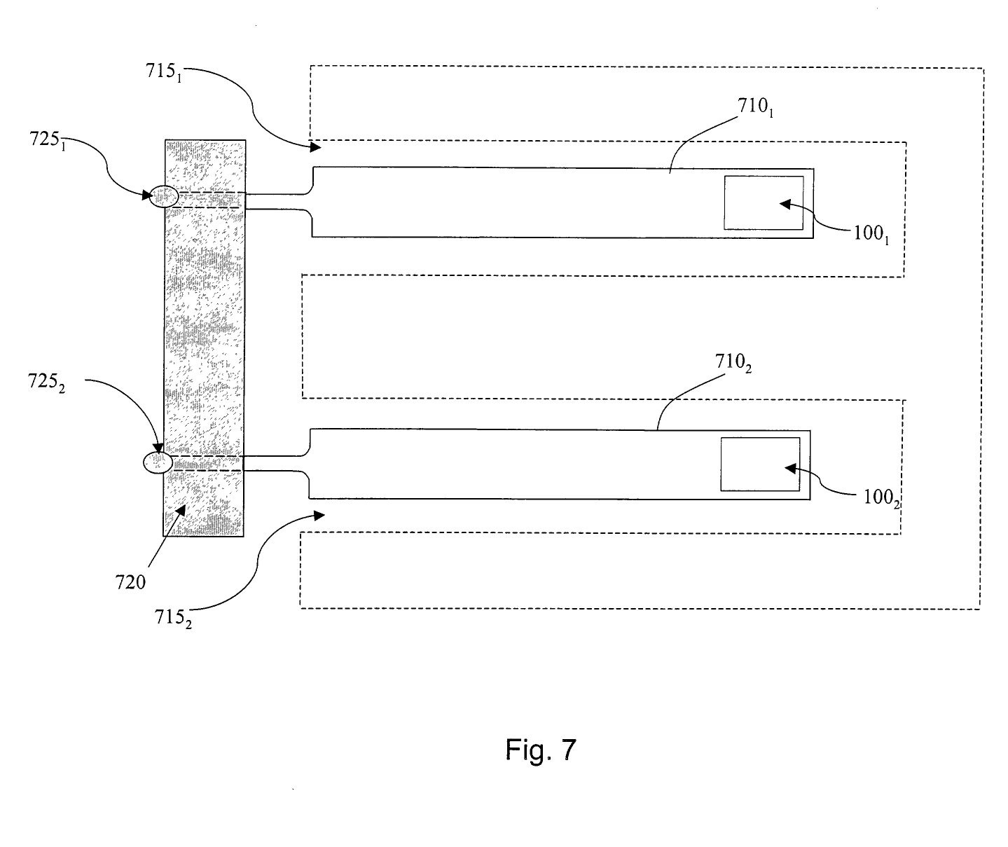

Referring to FIG. 7, two measurement devices 710₁, 710₂ in the cantilever configuration are shown within corresponding microfluidic channels 715₁, 715₂. The invention desirably utilizes sensors in pairs, relying on differential rather than absolute measurements to determine the extent of binding. This arrangement tolerates variations in sensing surface quality. In the dual-sensor configuration 700, each cantilever 710₁, 710₂ has an area at its tip comprising its own sensing surface 100₁, 100₂. The two cantilevers 710₁, 710₂ are attached to a mounting surface 720. The cantilevers each contain a respective terminal 725₁, 725₂ to facilitate connection to the measurement and driver circuitry described above and can be submerged to varying degrees into microfluidic channels 715₁, 715₂.

The paired cantilever configuration and other embodiments of the measuring apparatus have the advantage of facilitating performance of experiments with small reagent volumes, and also allow analytes to be deposited onto the sensor using pipettes. The small size and simplicity of the electrical circuitry can facilitate wireless electronic measurement.

In this differential dual sensor configuration different molecules can be pipetted into each cantilever and their respective electrical signatures can be compared. This configuration also facilitates one or more cantilever devices being submerged in microfluidic channels containing the analytes of interest. The probes bound to the sensing surface 100 may, for example, can be complementary oligonucleotide sequences. DNA molecules of interest 125 having an affinity for one of the probes will exhibit a much smaller affinity for the other probe, but the differential measurement of electrical signals independently resulting from binding to each probe facilitates measurement of binding with improved accuracy and reduced noise. A single base mismatch within 12 mer oligonucleotides can be distinguished using a differential detection technique with two sensors in parallel. In addition, the dual or single cantilever measurement devices can be sequentially scanned through different analyte containing channels to test the binding and hybridization of various molecules of interest with improvements in speed and sensitivity over conventional devices.

Having described certain preferred and exemplary embodiments of the invention, it will be apparent to those of ordinary skill in the art that other embodiments incorporating the concepts disclosed herein can be used without departing from the spirit and the scope of the invention. The described embodiments are to be considered in all respects only as illustrative and not limiting. Therefore, it is intended that the scope of the present invention be only limited by the following claims.

What is claimed is:

1. An apparatus for measuring biomolecular interaction, the apparatus comprising:
a. a sensing surface having an associated first charge;
b. a charge-regulating layer bound to the sensing surface, the charge-regulating layer having an associated second charge opposite to the first charge and presenting, in conjunction with the sensing surface, the second charge or a neutral charge on a net basis;
c. a probe bound to at least one of the sensing surface and the charge-regulating layer, the probe being complementary to and interacting with a molecule of interest; and
d. a measurement circuit, operatively connected to the sensing surface, for measuring interactions between the probe and the molecule of interest.

2. The apparatus of claim 1 wherein the second charge substantially neutralizes the first charge.

3. The apparatus of claim 1 further comprising a passivation layer at least over the sensing surface.

4. The apparatus of claim 1 wherein the first charge is a native negative charge and wherein the second charge is positive.

5. The apparatus of claim 3 wherein the passivation layer comprises a thermal oxide.

6. The apparatus of claim 4 wherein the charge-regulating layer comprises a charged polymer.

7. The apparatus of claim 6 wherein the charged polymer is polylysine.

8. The apparatus of claim 7 wherein the polylysine is electrostatically bound to the sensing surface.

9. The apparatus of claim 8 wherein the interaction of the probe and molecule of interest generates an associated electrical response in the measurement circuit, a magnitude of the electrical response being correlated with a degree of interaction.

10. The apparatus of claim 4 wherein the sensing surface is a gate of a field-effect transistor.

11. The apparatus of claim 4 wherein the sensing surface is silicon dioxide.

12. The apparatus of claim 4 wherein interaction between the probe and the molecule of interest alters a capacitance within the measurement circuit, the alteration being indicative of the interaction.

13. The apparatus of claim 4 wherein at least a portion of the capacitance arises between the sensing surface and an electrolyte solution immersing the sensing surface.

14. The apparatus of claim 4 wherein the interaction between the probe and the molecule of interest alters an electronic characteristic at the sensing surface, the alteration being indicative of the interaction.

15. The apparatus of claim 14 wherein the electronic characteristic is at least one of capacitance, conductance, impedance, resistance, current, voltage, and electric field intensity.

16. The apparatus of claim 4 wherein the measurement circuit comprises:
a. a charge-sensitive region underlying the sensing surface;
b. an electrolyte solution disposed on the sensing surface; and
c. a semiconductor region at least partially surrounding the charge-sensitive region, the sensing surface, charge-sensitive region, semiconductor region, and electrolyte solution forming at least one capacitor.

17. The apparatus of claim 16 wherein the measurement circuit further comprises a measurement module, a reference electrode, and a power source bridging the reference electrode and the semiconductor region.

18. The apparatus of claim 17 wherein the measurement circuit further comprises a sensor bias module, a current amplifier, a lock-in amplifier, and a data-acquisition module.

19. The apparatus of claim 16 wherein the charge-sensitive region is lightly doped silicon and the semiconductor is highly doped silicon.

20. The apparatus of claim 16 wherein the charge-sensitive region and at least a portion of the semiconductor region form a cantilever.

21. The apparatus of claim 20 wherein the cantilever is configured for insertion into a microfluidic channel.

22. The apparatus of claim 20 wherein the charge-sensitive region and at least a portion of the semiconductor region form a plurality of cantilevers electrically connected to facilitate differential measurements of the properties of molecules of interest.

23. The apparatus of claim 4 wherein the probe is a nucleic acid.

24. The apparatus of claim 4 wherein the probe is a protein nucleic acid.

25. The apparatus of claim 4 wherein the probe is a polypeptide.

26. The apparatus of claim 4 wherein the probe is a substrate interactive with a polypeptide.

27. The apparatus of claim 4 wherein the probe is an enzyme interactive with a substrate.

28. The apparatus of claim 4 wherein the probe is an antibody.

29. The apparatus of claim 4 wherein the probe is an antigen.

30. The apparatus of claim 4 wherein the measurement circuit is operatively connected to the sensing surface through only one electrical contact.

31. The apparatus of claim 4 wherein the apparatus is passivated to retain operational functionality notwithstanding cleaning.

32. The apparatus of claim 1 wherein the sensing surface has an area no greater than 50 μm².

33. Apparatus for measuring biomolecular interaction, the apparatus comprising:
a. a sensing surface comprising a probe complementary to and interacting with a molecule of interest; and
b. a measurement circuit, operatively connected to the sensing surface, for capacitively measuring interaction between the probe and the molecule of interest, interaction between the probe and the molecule of interest altering a capacitance within the measurement circuit, the alteration being indicative of the interaction.

34. A method of measuring biomolecular interaction, the method comprising the steps of:
a. providing a sensing surface having a native negative charge;
b. binding thereto a layer conferring to the sensing surface a neutral or positive charge;
c. binding a probe to at least one of the surface and the charge-conferring layer, the probe being complementary to and interacting with a molecule of interest; and
d. measuring interaction between the probe and the molecule of interest.

35. The method of claim 34 wherein the step of measuring interaction between the probe and the molecule of interest occurs within a low ionic strength solution environment.

36. The method of claim 34 further comprising the step of cleaning the sensing surface with strong cleaning agent.

37. The method of claim 34 wherein the strong cleaning agent comprises 3:1 H₂SO₄:H₂O₂.

38. The method of claim 34 further comprising
a. binding a new charge-conferring layer over the sensing surface and a previously applied charge-conferring layer;
b. binding a probe to at least one of the surface and the new charge-conferring layer, the probe being complementary to and interacting with a molecule of interest; and
c. measuring interaction between the probe and the molecule of interest.

39. A method of measuring biomolecular interaction, the method comprising the steps of:
a. providing a sensing surface comprising a probe complementary to and interacting with a molecule of interest; and
b. capacitively measuring interaction between the probe and the molecule of interest.

40. The method of claim 39 further comprising the steps of:
a. providing an additional sensing surface comprising an additional probe complementary to and interacting with the molecule of interest;
b. capacitively measuring interaction between the additional probe and the molecule of interest; and
c. assessing an extent of binding through differential analysis of the interaction with the probe and the interaction with the additional probe.

41. The method of claim 39 wherein the step of measuring interaction between the probe and the molecule of interest occurs within a low ionic strength solution environment.

42. The method of claim 39 further comprising the step of cleaning the sensing surface with a strong cleaning agent.

43. The method of claim 42 wherein the strong cleaning agent comprises 3:1 H₂SO₄:H₂O₂.

44. A method of measuring biomolecular interaction, the method comprising the steps of:
a. providing a semiconductor sensing surface;
b. removing any oxide on the surface;
c. exposing the surface to a medium promoting growth of a thin insulating layer thereover;
d. disposing a probe over the thin insulating surface; and
e. measuring interaction between the probe and the molecule of interest.

45. The method of claim 44 wherein the medium comprises 3:1 H₂SO₄:H₂O₂.

46. The method of claim 44 wherein the thin insulating surface is a chemical oxide.
